(19) United States
(12) Patent Application Publication
(10) Pub. No.: US 2004/0138429 A1
(43) Pub. Date: Jul. 15, 2004

(54) Bioemulsifier production by Acinetobacter strains isolated from healthy human skin

(75) Inventors: J. R. Patil, Pune (IN); B. A. Chopade, Pune (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/629,309

(22) Filed: Jul. 29, 2003

Related U.S. Application Data
(60) Provisional application No. 60/399,498, filed on Jul. 30, 2002.

(51) Int. Cl. C07K 14/195; A61K 38/16

(52) U.S. Cl. 530/395; 435/69.1; 435/252.3; 435/320.1; 514/8

(57) ABSTRACT

A bioemulsifier has protein content 50.5%, polysaccharide content 43% and lipid content 3.8%.

### OTHER REFERENCES

1. Baumann, P. (1968) Isolation of Acinetobacter from soil and water. Journal of Bacteriology 96, 39-42.

2. Berlau, J., Aucken, H., Malnick, H. and Pitt, T. (1999). Distribution of Acinetobacter species on skin of healthy humans. European Journal of Clinical Microbiology and Infectious Diseases 18, 179-183.

3. Bouvet, P. J. M. and Grimont, P. A. D. (1986) Taxonomy of the genus Acinetobacter with the recognition of Acinetobacter baumannii sp. nov., Acinetobacter haemolyticus sp. nov., Acinetobacter johiisonii sp. nov., and Acinetobacter junii sp. nov. and emended descriptions of Acinetobacter calcoaceticus and Acinetobacter iwoffli. International Journal of Systematic Bacteriology 36, 228-240.

4. Bouvet, P. J. M. and Grimont, P. A. D. (1987) Identification and biotyping of clinical isolates of Acinetobacter. Annals of Institute of Pasteur Microbiology 138, 569-578.

5. Budavari, S., O'Neil, M. J., Smith, A., Heckelman, P. E. and Kinneary, J. F. (1996) The Merck Index—an encyclopedia of chemicals, drugs and biologicals. 12th edn. Whitehouse Station, N.J., USA: Merck & Co. Inc.

6. Chu, Y. W., Leung, C. M., Houang, E. T. S., Ng, K. C., Leung, C. B., Leung, H. Y. and Cheng, A. F. B. (1999). Skin carriage of acinetobacters in Hong Kong. Journal of Clinical Microbiology 37, 2962-2967.

7. De Vries, R. J. (1987) Industrial uses of palm oil. Critical Reviews in Applied Chemistry 15,92-97.

8. Downing, D. T., Stewart, M E. and Strauss, J. S. (1999) Lipids of the epidermis and the sebaceous glands. In Fitzpatrick's Dermatology in General Medicine ed. Freedberg, I. M., Eisen, A. Z., Wolff, K., Austen, K. F., Goldsmith, L. A., Katz, S. A. and Fitzpatrick, T. B. pp. 144-155. SA: McGraw-Hill Inc.

9. Dubois, M., Gilles, K. A., Hamilton, J. K., Rebers, P. A. and Smith, F. (1956). Colorimetric method for determination of sugars and related substances. Analytical Chemistry 28, 350-356.

10. Eigen E, and Simone A J. (1986) Control of dental plaque and caries. U.S. Pat. No. 4,619,825.

11. Foght, J. M., Gutnlick, D. L. and Westlake, D. W. S. (1989) Effect of emulsan on biodegradation of crude oil by pure and mixed bacterial cultures. Applied and Environmental Microbiology 55, 36-42.

12. Gutnick, D. L. and Rosenberg, E. (1977) Oil tankers and pollution: a microbial approach. Annual Review of Microbiology 31, 379-396.

13. Gutnick, D. L. and Minas, W. (1987) Perspectives on microbial surfactants. Biochemical Society Transactions 22s-35S.

14. Gutnick D. L., Nestass E., Rosenberg E., and Sar N. (1989). Bioemulsifier production by Acinetobacter calcoaceticus strains. U.S. Pat. No. 4,883,757.

15. Hayes M. E. (1989) Bioemulsifier-containing personal care products for topical application to dermopathologic conditions of the skin and scalp. U.S. Pat. No. 4,870,010.

16. Hayes M E. (1991) Personal care products containing bioemulsifiers. U.S. Pat. No. 4,999,195.

17. Horie, K. (1988) Manufacture of stable oil-in-water emulsions for cosmetics, pharmaceuticals, and foods. JP 63221834 A2.

18. Hoshizaki, S. and Suzuki, T. (1978) Cosmetics containing castor oil glyceride esters. JP53148543.

19. Juni, E. (1972) Interspecies transformation of Acinetobacter: genetic evidence for a ubiquitous genus. Journal of Bacteriology 112, 917-931.

20. Kaplan, N. and Rosenberg, E. (1982) Exopolysaccharide distribution of and bioemulsifier production by Acinetobacter calcoaceticus BD4 and BD413. Applied and Environmental Microbiology 44, 1335-1341.

21. Kaplan, N., Zosim, Z. and Rosenberg, E. (1987) Reconstitution of emulsifying activity of Acinetobacter calcoaceticus BD4 emulsan by using pure polysaccharide and protein. Applied and Environmental Microbiology 53, 440-446.

22. Lowry, O. H., Rosenbrough, N. J., Farr, A. L. and Randall, R. (1951). Protein measurement with the Folin phenol reagent. Journal of Biological Chemistry 193, 265-275.

23. Monreal, J. and Reese, E. T. (1969) The chitinase of Serratia marcescens. Canadian Journal of Microbiology 15, 689-696.

24. Navon-Venezia, S., Zosim, Z., Gottlieb, A., Legmann, R., Carmeli, S., Ron, E. Z. and Rosenberg, E. (1995) Alasan, a new bioemulsifier from Acinetobacter raclioresistens. Applied and Environmental Microbiology 61, 3240-3244.

25. Pines, O. and Gutnick, D. (1986) Role of emulsan in growth of Acinetobacter calcoaceticus RAG-I on crude oil. Applied and Environmental Microbiology 51, 661-663.

26. Prosperi G., Camilli M., Crescenzi F., Fascetti E., Porcelli F., and Sacceddu P. (2000). Lipopolysaccharide biosurfactant. U.S. Pat. No. 6,063,602.

27. Reddy, P. G., Singh, H D., Pathak, M. G., Bhagat, S. D. and Baruah, J. N. (1983). Isolation and functional characterization of hydrocarbon emulsifying and solubilizing factors produced by a Pesudomonas species. Biotechnology and Bioengineering 25, 387-401.

28. Rosenberg, E., Zuckerberg, A., Rubinovitz, C. and Gutnick, D. L. (1979a) Emulsifier of Arthrobacter RAG-1: isolation and emulsifying properties. Applied and Environmental Microbiology 37, 402-408.

29. Rosenberg, E., Peery, A., Gibson, D. T. and Gutnick, D. L. (1979b) Emulsifier of Arthrobacter RAG-1: specificity of hydrocarbon substrate. Applied and Environmental Microbiology 37, 409-413.

30. Rosenberg, M., Gutnick, D. and Rosenberg, E. (1980) Adherence of bacteria to hydrocarbons: a simple method for measuring cell-surface hydrophobicity. FEMS Microbiology Letters 9, 29-33.

31. Rosenberg, E. (1986) Microbial surfactants. Critical Reviews in Biotechnology 3, 109-132.

32. Rosenberg, E., and Ron E. Z. (1998). Bioemulsifiers. U.S. Pat. No. 5,840,547.

33. Rubinovitz, C., Gutnick, D. L. and Rosenberg, E. (1982) Emulsan production by Acinetobacter calcoaceticus in the presence of chloramphenicol. Journal of Bacteriology 152,126-132.

34. Sar, N. and Rosenberg, E. (1983) Emulsifier production by Acinetobacter calcoaceticus strains. Current Microbiology 9, 309-314.

35. Seifert, H., Dijkshoorn, L., Gerner-Smidt, P., Pelzer, N., Tjenberg, I. and Vaneechoutte, M. (1997). Distribution of Acinetobacter species on human skin: comparison of phenotypic and genotypic identification methods. Journal of Clinical Microbiology 35, 2819-2825.

36. Shabtai, J., Pines, O. and Gutnick, D. (1985) Emulsan: a case study of microbial capsules as industrial products. Development in Industrial Microbiology 26, 291-307.

37. Shabtai, Y. and Gutnick, D. L. (1985) Exocellular esterase and emulsan release from the cell surface of Acinetobacter calcoaceticus. Journal of Bacteriology 161, 1176-1181.

38. Shi, Y. P., and Li, Z. Y. (1989) A bacterial liopolysaccharide emulsifier. Chinese Journal of Biotechnology 5, 231-140.

39. Strauss, I. S., Pochi, P. E. and Downing, D. T. (1975) Skin lipids and acne. Annual Review of Medicine 26, 27-32.

40. Szepessy, G., Kaposi, M. S., Mohr, T., Horvath, K., Furjes, J. and Furjes, D. (1990) Cosmetics containing vegetable oils and vitamins. HU 51890 A2.

41. Zajic, I. E. and Panchal, C. J. (1976) Bioemulsifiers. Critical Reviews in Microbiology 5,39-66.

42. Zosim, Z., Gutnick, D. and Rosenberg, E. (1982) Properties of hydrocarbon-in-water emulsions stabilized by Acinetobacter RAG-1 emulsan. Biotechnology and Bioengineering 24, 281-292.

43. Zuckerberg, A., Diver, A., Peeri, Z., Gutnick, D. L. and Rosenberg, E. (1979) Emulsifier of Arthrobacter RAG-1: chemical and physical properties. Applied and Environmental Microbiology 37, 414-420.

In recent years, interest has been growing in search for novel bioemulsifiers. Many bacterial genera including Acinetobacter have been reported to produce bioemulsifiers. The present study aims to screen Acinetobacter isolates from healthy human skin for bioemulsifier production.

Acinetobacter junii SC14 (NCIM 5150) produced maximum bioemulsifier in presence of almond oil during stationary growth phase at 37OC and pH 7.2. Partially purified, non-dialysable bioemulsifier from SC14 was a proteoglycan. Protein and polysaccharide fractions resulted in 95.2% reconstitution of emulsification activity. Role of esterase in release of cell-bound emulsifier and contribution of capsular polysaccharide to emulsification activity were observed.

Acinetobacter strains from human skin exhibited better emulsification activity than that by bum wound or soil isolates owing to the inherent differences in chemical microenvironment of their habitats.

Investigation of skin commensals, especially acinetobacters, would lead to discovery of novel bioemulsifiers with interesting properties. Attempts of screening and strain improvement directed towards skin commensals will open up new avenues for strains producing bioemulsifier on commercial scale.

### BACKGROUND OF THE INVENTION

Growth of microorganisms on water insoluble carbon sources is often accompanied by emulsification of the water insoluble substrate in the culture medium by virtue of production of bioemulsifiers (Zajic and Panchal 1976; Rosenberg 1986). Many amphipathic molecules produced by living cells possess surface-active characteristics observed in form of lowering of interfacial tension at the oil-water interface which leads to formation of microemulsions (Gutnick and Minas 1987). Bioemulsifiers are an example of such surface-active agents of microbial origin. Bioemulsifiers are amphipathic molecules playing a vital role in microbial growth on hydrophobic substrates. These bioemulsifiers find potential applications in cosmetic, food, agricultural and pharmaceutical industries (Rosenberg 1986). Moreover, health hazards of oil spills can also be minimized through bioremediation employing specific microorganisms (Gutnick and Rosenberg 1977). Among a variety of bacterial genera studied, production of extracellular emulsifiers has been observed to be a phenomenon of common occurrence among the members of genus Acinetobacter (Rosenberg 1986). Emulsan, an extracellular polyanionic bioemulsifier produced by Acinetobacter calcoaceticus RAG-1 has been studied in great detail (Foght et al. 1989; Rubinovitz et al. 1982; Pines and Gutnick 1986; Zuckerberg et al. 1979; Rosenberg et al. 1979a; Rosenberg et al. 1979b; Zosim et al. 1982). Many strains of Acinetobacter isolated from soil, mud, marine water, fresh water etc. have been reported to produce bioemulsifiers (Sar and Rosenberg 1983; Foght et al. 1989). Moreover, few reference strains, viz, Ac. calcoaceticus BD4 and BD413, ATCC 17294, ATCC 17906 were also investigated for emulsifier production (Kaplan and Rosenberg 1982; Kaplan et al. 1987).

Few patents have been awarded for bioemulsifier production by various Acinetobacter strains (Gutnick et al. 1989; Rosenberg and Ron 1998; Prosperi et al. 2000). Moreover, various health care products, such as topical application for skin, toothpaste and mouthwash, and personal care products, consisting of bioemulsifiers produced by Acinetobacter strains have also been patented (Hayes 1989; Hayes 1991; Eigen and Simone 1986). However, there is no report of bioemulsifier production by Acinetobacter strains from human skin. Acinetobacters have been isolated from skin of around 40% human population with recovery of 7 to 18 genospecies but the dominant isolates included Ac. iwoffli, Ac. junii, Ac. johnsonii, Ac. calcoaceticus etc. (Seifert et al. 1997; Berlau et al. 1999; Chu et al. 1999). Presence of numerous hydrophobic substances on human skin in the form of Skin Surface Lipids (SSL) (Strauss et al. 1975; Downing et al. 1999) necessitates that the skin microflora be able to resist the inhibitory action of SSLs and if possible, be capable of metabolizing these substrates. In view of this background, bioemulsifier production by Acinetobacter strains isolated from healthy human skin was studied. Apart from these naturally occurring moieties, considerable fraction of oils can be contributed to the skin microenvironment in form of cosmetics. One can find substantial documentation of use of oils, viz, almond oil, castor oil, olive oil, palm oil etc., in the manufacture of cosmetics, especially the skin-care products (Hoshizaki and Suzuki 1978; De Vries 1987; Horie 1988; Szepessy et al. 1990). These facts prompted us to screen skin Acinetobacter strains for bioemulsifier production. The present paper describes bioemulsifier production by Acinetobacter strains from healthy human skin, partial purification and characterization of the bioemulsifier, and study of effect of physico-chemical factors on it's production and activity.

### OBJECTS OF THE INVENTION

The main object odf the present invention is to provide a bioemulsifier production by acinetobacter strains isolated from healthy human skin.

Another object of the present invention to provide a bioemulsifier having peak esterase activity cell associated of order of 61.3% and 38.6% activity was secreted into the fermentation medium.

Yet another object of the present invention to provide the bioemulsifier wherein increase in bioemulsifier concentration from 0.5 ml to 3 ml against a fixed volume of 6 ml almond oil resulted in a reduction of viscosity of almond oil by 40.3%.

Further object of the present invention is to provide a bioemulsifier wherein the almond oil and water emulsion maintains its stability up to 90% up to 6 days at 37° C.

In yet another object of the present invention is to provide a bioemulsifier wherein the bioemulsifier retains 35% stability after 140 hours at 10° C.

Still another object of the present inventio is to provide a bioemulsifier which is useful for preparing stable cosmetics (skin care product) and stable pharmaceutical ointment preparations.

Another object of the present invention is to provide a process for preparation of a bioemulsifier by acinetobacter strains isolated from healthy human skin.

### SUMMARY OF INVENTION

A bioemulsifier is produced by Acinetobacter junii SC14 (NCIM 5150). The strain was isolated from healthy human skin with Baumann's enrichment method. The strain was identified as Acinetobacter by genetic and physiological studies; the colonies showing nonmotile, oxidase-negative coccobacilli. Genomic DNA of this strain transformed Acinetobacter calcoaceticus BD413 trpE27 to prototrophy. Almond oil was emulsified to the maximum extent.

### DETAILED DESCRIPTION OF THE INVENTION

Source and Biotyping of Bacterial Strains Used

The isolate was obtained after enrichment of respective samples in Baumann's enrichment medium (Baumann 1968). Identification of the isolate up to genus level was carried out as per the chromosomal DNA transformation assay (Juni 1972). Delineation of the isolate into various genospecies was performed as per the biochemical scheme recommended by Bouvet and Grimont (1986,1987). The control strains used included Acinetobacter calcoaceticus ATCC 33305, Ac. calcoaceticus EBF 65/65 C426, Ac. calcoaceticus EBF 65/65 C4169, Ac. calcoaceticus BD4, Ac. calcoaceticus BD413, Ac. calcoaceticus MTCC 127, Ac. calcoaceticus MTCC 1271, Ac. calcoaceticus MTCC 1425, Ac. lwoffli MTCC 496, Escherichia coli MTCC 68, E. coli K12 HB101, E. coli K12 DH5α, E. coli K12 JM103, Pseudomonas fluorescens MTCC 740, Ps. putida MTCC 1313, and Staphylococcus aureus MTCC 740.

Oils Used as Substrates

Almond oil from Hamdard (WAKF) Laboratories, Ghaziabad, UP, India; castor oil from Shree Krishna Pharmacy, Mumbai, India; olive oil from Campbell Agro Mfg Industries Pvt. Ltd, Mumbai, India and palm oil from Chakan Oil Mills, Chakan, India were used as substrates during fermentation. All oils were of analytical grade.

Bioemulsifier Production

The mineral salt medium (BNP) used (Foght et al. 1989) consisted of (1-1 solution) 0.5 g K2HPO₄, 1 g NH4Cl, 2 g Na2SO4, 2 g KNO3, 0.2 g MgSO4,7H₂O, and 0.002 g FeSO4,7H2O. The medium was supplemented with 1 g 1-1 peptone. The pH of the medium was adjusted to 7.2. Bioemulsifier production was carried out in 50 ml of above medium in a 250 ml Erlenmeyer flask at 37OC with shaking at 150 rev min-1. Soil isolates were grown at 30OC. Bacterial growth was initiated by introduction of 1% (v/v) inoculum growing exponentially in the same medium. Desired quantity of respective test oil (v/v) was added to the cooled fermentation medium after autoclaving. Samples withdrawn at 5 h interval were first used to record the culture density at 660 nm and then used for the emulsification assay.

Emulsification Assay

Three ml cell-free culture broth or a suitably diluted emulsifier preparation was mixed with 0.5 ml test oil, vortexed vigorously for 2 min and incubated at 37OC for 1 h. Absorbance of the aqueous phase was then recorded at 400 nm. The absorbance maxima was arrived at after scanning the entire visible light spectrum (UV-1601 Shimadzu Corporation, Japan). The blank was prepared similarly with sterile production medium replacing the cell-free culture broth or the emulsifier aliquot. An absorbance of 0.010 units at 400 nm multiplied by dilution factor, if any, was considered as one unit of emulsification activity per ml (EU ml-1).

Stability of Emulsion

After 1 h incubation of the vortexed mixture of test oil and emulsifier at 37OC, the absorbance of aqueous layer at 400 nm was recorded. One set was incubated at 37OC and the other at 10OC. Absorbance of aqueous layer was noted every 20 h (Zosim et al. 1982).

Effect of Physico-Chemical Factors on Bioemulsifier Production and Activity

Effect of temperature (20, 30, 40, and 500C) on bioemulsifier production was studied by growing the test strain at the respective temperature in a shaking water bath at 150 rev mind and then checking the culture supernatant for oil emulsification as per the method described above. Effect of temperature on bioemulsifier activity was studied by incubating the vortexed cell supernatant and oil mixture at the respective temperatures for 1 h and then recording the absorbance of the aqueous layer at 400 nm. Effect of pH (4, 5, 6, 7, 8 and 9) on bioemulsifier production and activity was studied similarly. The optimum temperature of 37OC was used in all experiments hereafter unless stated otherwise. The fermentation medium was supplemented with required quantities of one of the three test salts (NaCl, CaCl2, MgCl2) to study the effect of salts on bioemulsifier production. Effect of salts on bioemulsifier activity was tested by incubating the bioemulsifier sample in presence of required quantity of test salt at 37OC for 1 h and then using it in the standard emulsification assay.

Effect of Inducer Oil on Bioemulsifier Production

Growth of Ac. junii SC14 was allowed in BNP medium supplemented with 1% of test oils separately. The resulting cell-free supernatants were assayed for emulsification of each test oil according to the emulsification assay described above.

Partial Purification of Bioemulsifier

Culture of Acinetobacter junni SC14 from healthy human skin has been deposited at National collection of Industrial Microorganisms (NICM). The NCIM no for this culture is 5150Ac. junii SC14 (henceforth referred to as SC14) producing maximum levels of bioemulsifier was selected for further study. The cell free culture broth obtained by centrifugation at 8000 rev min-I for 20 min was mixed with three volumes of chilled acetone and incubated at 4OC for 15 h. The brown precipitate was collected by centrifugation at 10,000 rev min-1 for 30 min and dissolved in minimum volume of sterile distilled water (pH 7). This solution was then dialyzed (Seamless cellulose tubing, width 40 mm, diameter 25 mm, retaining most proteins of molecular weight 12,000 or greater, Sigma Aldrich Chemie, Gmbh, Steinheim, Germany) extensively against sterile distilled water at 10OC for 48 h. The distilled water in dialysis container was replaced every 10 h. The dialysate was then frozen at −20OC and lyophilised. This bioemulsifier preparation was stored in airtight glass vials at room temperature (30OC).

Chemical Analysis of Bioemulsifier

A portion of the partially purified bioemulsifier was used for chemical analysis. Protein content was assayed using the method described by Lowry et al (1951) with bovine serum albumin as standard. Carbohydrates were quantitated according to the protocol proposed by Dubois et al (1956). Reducing sugar content was estimated using the dinitro salicylic acid method (Monreal and Reese 1969) with glucose as a standard. Extraction and quantitation of lipids was performed as per the method described by Reddy et al (1983).

Determination of Viscosity

Different aliquots (0.25 to 3.0 ml) of solution of the partially purified bioemulsifier (5 mg ml-1) were used to emulsify fixed volume of almond oil (6 ml). The viscosity of these emulsified oil samples (6 ml) was recorded using a standard viscometer at 25OC. Readings were taken in duplicate and the average was reported. Unemulsified almond oil was used as the control.

Determination of Esterase Activity

Quantification of the esterase activity was performed according to the colorimetric assay protocol described by Shabtai and Gutnick (1985) with p-nitrophenyl acetate as the substrate. Cell pellet as well as the cell-free supernatant was assayed for esterase activity after every 10 h during the 60 h long fermentation.

Reconstitution of Emulsification Activity

On the basis of chemical composition of the partially purified emulsifier, the components were isolated. The protein and polysaccharide fractions from the bioemulsifier (50 mg) and the cell-free supernatant (300 ml) were isolated. Protein fraction of the purified bioemulsifier was prepared by hot phenol treatment. Extracellular protein was obtained by 60% ammonium sulfate precipitation of the cell-free culture broth. The polysaccharide fraction from purified bioemulsifier was isolated by water extraction of phenol phase while the capsular polysaccharide was obtained by acetone precipitation of the homogenized culture supernatant (Kaplan et al. 1987). These fractions were then checked for their ability to reconstitute the almond oil emulsification property, individually or in combination, according to the emulsification assay described above.

Cell-Surface Hydrophobicity Assay

Cell surface hydrophobicity of the test strains was determined using hexadecane and almond oil (Rosenberg et al. 1980) with slight modification. Volume of test hydrocarbon used ranged from 0.1 to 1.6 ml. Incubation of the culture and hydrocarbon was carried out at 37OC instead of 30OC except in case of the soil isolate. Ac. baumannii SB-1 isolated from burn wound (referred to as SB-1) and Ac. calcoaceticus GS1LB isolated from soil (referred to as GS1LB) were also tested. Escherichia coli MTCC 68 (referred to as EC68) and Pseudomonas aeruginosa MTCC 1223 (referred to as PA1 223) were used as controls.

Results

Biotyping of Acinetobacter strains

The skin isolate was identified to be Ac. junii. The chromosomal DNA transformation assay was useful in confirming the generic identity of the isolate while the biochemical scheme proposed by Bouvet and Grimont (1986, 1987) helped us in delineation of the isolate into various Acinetobacter genospecies.

Bioemulsifier Production by Acinetobacters From Human Skin

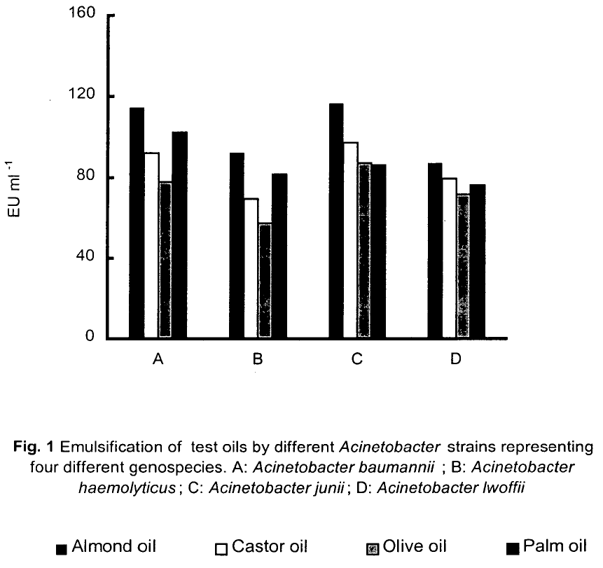

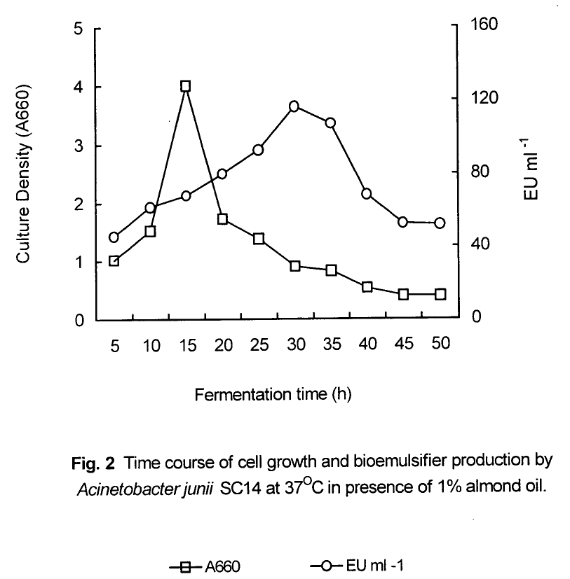

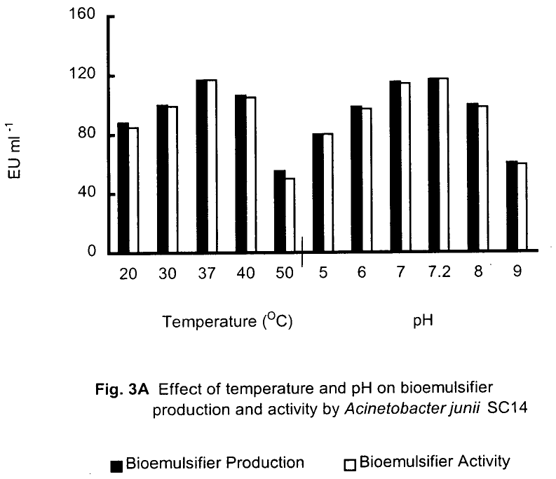

Ac. junii SC14 isolated from healthy human skin exhibited maximum bioemulsifier production followed by Ac. baumannii (FIG. 1). Ac. haemolyticus emulsified olive oil to the minimum extent while palm oil was minimally emulsified by Ac. junii. In general, almond oil was emulsified to the maximum extent while palm oil was least emulsified. SC14 emulsified almond oil to the maximum extent. The kinetics of bioemulsifier production and growth pattern of SC14 is shown in FIG. 2. Fermentation for 30 h under given set of conditions led to production of 116.6 EU ml-1 in presence of 1% almond oil.

Stability of Emulsion

The almond oil-in-water emulsion was observed to be highly stable. Emulsion maintained 90% of it's stability up to 6 days at 37OC after which phase separation was observed. However, 30% loss of stability was noted within 20 h at 10OC. Emulsion retained only 35% stability after 140 h at 10OC.

Effect of Physico-Chemical Factors

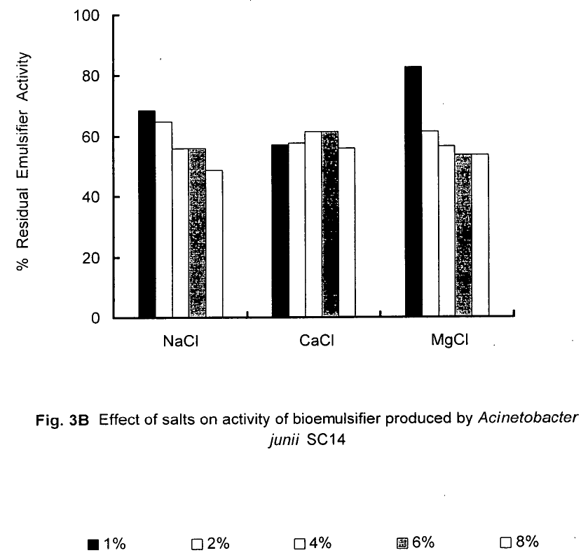

In case of SC14, fermentation at 37OC and pH 7.2 in presence of 0.5% NaCl was observed to yield the highest levels of bioemulsifier (FIG. 3A). It was found that at 50OC, more than 50% bioemulsifier activity was inhibited making it comparatively less thermostable. Incubation at 37OC and pH 7.2 also were optimum for the emulsification activity. However, 1% concentration of NaCl, CaCl2, and MgCl2, used separately, caused 31.3, 43.6, and 17.3% inhibition of the emulsification activity, respectively (FIG. 3B).

Substrate Specificity of Bioemulsifier

Among the four oils tested, almond oil served as the best substrate for emulsification for all the strains tested (FIG. 1). Palm oil was emulsified to the least extent. Also maximum emulsification of almond oil was observed irrespective of the oil used during fermentation (Table 1) with the exception of castor oil. Strain SC14 was observed to tolerate up to 40% almond oil but the optimum bioemulsifier production took place in presence of 18% almond oil (427.8 EU ml-1).

Partial Purification of Bioemulsifier

The partially purified bioemulsifier obtained from cell-free supernatant of SC14 culture grown in presence of 18% almond oil exhibited 1759.8 EU ml-1. The bioemulsifier yield was 3.9 g 1-1 under given set of conditions. Chemical analysis of this bioemulsifier revealed that protein (50.5%) was the major constituent followed by polysaccharide (43%). It was noted that 88.7% of the polysaccharide comprised of reducing sugars. A minor fraction of lipid (3.8%) was also detected in the bioemulsifier. Thus proteoglycan nature of the bioemulsifier produced by SC14 is evident from it's chemical composition.

Esterase Activity

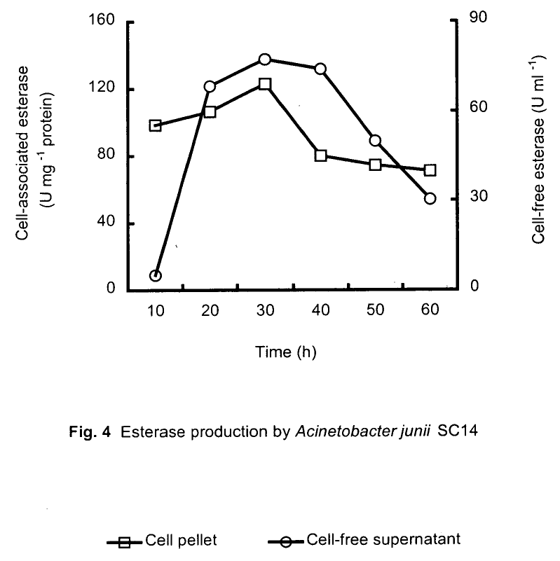

Kinetics of esterase production by SC14 is shown in FIG. 4. The highest esterase activity (122.8 U mg-1 of protein) was found to be cell-associated at 30 h at which time the extracellular broth also showed esterase activity (77.4 U ml-1) during the 60 h fermentation. Notably 61.3% of the peak esterase activity was observed to be cell-associated while only 38.6% activity was secreted into the fermentation medium. Moreover, it was observed that throughout the fermentation period, significant esterase activity was observed to be associated with the cells while only a minor fraction was detected in the fermentation broth.

Viscometry

Increase in bioemulsifier concentration from 0.5 ml to 3 ml against a fixed volume of 6 ml almond oil resulted into reduction in viscosity of almond oil by 40.3%.

Cell-Surface Hydrophobicity

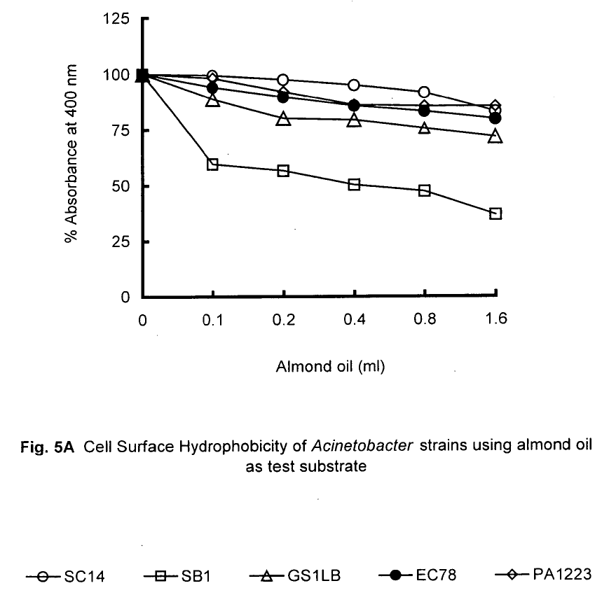

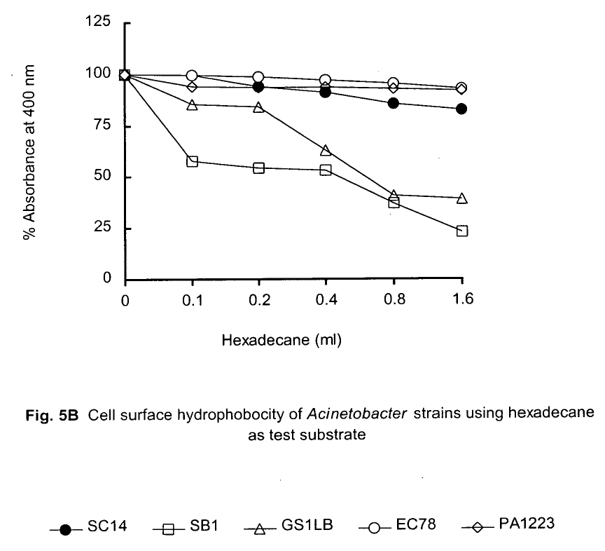

Degree of adherence of SC14, SB1, 1LB, EC78, and PA1223 to almond oil and hexadecane is shown in FIGS. 5A and 5B. Ac. junii SC14 showed lower affinity towards almond oil as well as hexadecane as compared to it's counterparts from burn wound and soil. Even the control strains of E. coli and Ps. aeruginosa had higher affinity for almond oil and hexadecane than SC14. Thus the cell surface of SC14 appeared to be less hydrophobic than that of the other test and control strains.

Reconstitution of Bioemulsifier Activity

Table 2 summarises the % emulsification activity reconstituted in presence of the given fractions using almond oil as the substrate. The fractions did show emulsification activity independently but the activity was enhanced when all the fractions were present together. Polysaccharide fractions from the bioemulsifier and extracellular broth showed lower emulsifying activity than the extracellular protein and the bioemulsifier protein. It should be noted that a prominent minicapsule was evident during the logarithmic growth phase of SC14 while only traces of the capsule were seen on cell surface during the stationary phase.

Acinetobacter junii SC14 from human skin exhibited the maximum bioemulsifier production. Peak bioemulsifier production by SC14 was observed at 30 h at 37OC under given set of conditions. This period coincides with the mid-stationary growth phase of the strain. Fermentation for an extended period of 30 h caused sharp decline in the bioemulsifier production to less than 50% of the peak value. Rosenberg et al (1979a) have also reported maximum emulsan production by Ac. calcoaceticus RAG-1 during the stationary growth phase. Bioemulsifier production by Ac. calcoaceticus RAG-1 can be attributed to it's metabolic requirement of ability to grow on various hydrophobic substances prevalent in marine environment. Similarly, the human skin does receive many hydrophobic compounds in the form of SSLs which are a mixture of triglycerides, fatty acids, wax esters, squalene, cholesterol and cholesterol esters (Strauss et al. 1975; Downing et al. 1999). Human skin is also exposed to exogenous oily compounds in form of skin-care cosmetics which contain almond oil, castor oil, olive oil and palm oil (Hoshizaki and Suzuki 1978; De Vries 1987; Horie 1988; Szepessy et al. 1990). In this way, presence of various hydrophobic substances on human skin can be a predisposing factor for the emulsification ability of the resident microbes.

Chemical composition of the test oils (Budavari et al. 1996) was also taken-into consideration during study of the bioemulsifier. All the test oils comprised mainly of three fatty acids, viz, oleic acid, linoleic acid and palmitic acid, in varying proportions. Oleic acid and linoleic acid are unsaturated while palmitic acid, stearic acid and arachidic acid are the saturated ones. Accordingly, almond oil and castor oil display a higher degree of unsaturation as compared to olive and palm oil. Still almond oil was attacked most readily by the test strains while palm oil with the least degree of unsaturation was not easily emulsified.

Optimum temperature for bioemulsifier production was found to be 37OC which is also suggestive of it's potential role in growth of skin bacteria. More than 50% loss of activity of the present bioemulsifier at 50OC is contrary to the observation of heat activation of alasan (Navon-Venezia et al. 1995). Emulsan is reported to be maximally produced and active at 30OC since the producer strain, Ac. calcoaceticus RAG-1, is an environmental isolate (Rosenberg et al. 1979a). Slightly alkaline pH of 7.2 was found to be optimum for bioemulsifier production while a slightly acidic pH of 6.5 allowed maximum bioemulsifier activity. Human skin is generally acidic (pH 5.6 to 5.8) but the present bioemulsifier showed a near neutral pH optimum. This variation can be due to the physicochemical differences between the in vivo and in vitro systems. In case of alasan produced by Ac. raclioresistens KA53 (Navon-Venezia et al. 1995), activity was observed over a wide pH range of 3.3 to 9.2 with an optimum at pH 5. The bioemulsifier investigated in the present study exhibited 80 EU ml-1at pH 5. However, at pH 9, about 48% of the bioemulsifier production and activity was inhibited. It indicates that higher bioemulsifier production and activity are attainable in the acidic pH than the alkaline pH. Among the three salts tested, CaCl2 caused stronger inhibition of bioemulsifier activity than NaCl and MgC12. In presence of 2% CaCl2 and MgCl2, bioemulsifier production remained unaffected while the activity was inhibited. The same percentage of NaCl resulted in 30% inhibition of bioemulsifier activity. In case of alasan produced by Ac. radioresistens KA53, magnesium ions stimulated the activity below and above the pH optimum (Navon-Venezia et al. 1995). Overall, none of the test salts showed considerable increase in the production and or activity of the bioemulsifier by SC14.

The almond oil in water emulsion was found to retain 90% stability at 37OC till 120 h but lost 70% stability at 10OC within 40 h. High emulsion stability at room temperature is preferable as it alleviates the need of storage at low temperature for preservation and also extends the shelf life of the product. Moreover, emulsification of almond oil by the partially purified bioemulsifier resulted in considerable reduction in the oil viscosity, which is a desired property for an emulsifier.

Cell surface hydrophobicity assay revealed that SC14 had a slightly higher affinity for hexadecane than almond oil. Accordingly, higher emulsification of hexadecane (204 EU ml-1) was observed than that of almond oil (116.6 EU ml-1). However, Ps. aeruginosa with lower affinity for hexadecane exhibited better emulsification of hexadecane (232 EU ml-1) as compared to that of the almond oil (91.9 EU ml-1). Ac. baumannii SB1 had greater affinity for almond oil as well as hexadecane than that by SC14 but showed comparatively lower emulsification (106.3 EU ml-1 and 116.6 EU mil-1 for almond oil by SB1 and SC14, respectively; 116.2 EU ml-1 and 204 EU ml-1 for hexadecane by SB1 and SC14, respectively). Thus SC14, despite it's lower affinity to almond oil, can utilize it in a more efficient fashion than SB1. In an earlier report, Ps. aeruginosa PAS 279 has been reported to display a similar pattern (Rosenberg et al. 1980). Thus the correlation between the affinity to hydrophobic substrate and the degree of it's utilization remains to be established.

Polysaccharide fraction of the proteoglycan bioemulsifier consisted of 88.7% reducing sugars. Emulsan produced by RAG-1 has been shown to be an extracellular noncovalent complex of a lipopolysaccharide and a protein (Zuckerberg et al. 1979). A lipopolysaccharide emulsifier by Acinetobacter has also been reported (Shi and Li 1989). However, alasan produced by Ac. radioresisteiis KA53 consists of an alaninie-rich heteropolysaccharide and a protein (Navon-Venezia et al. 1995). Yield of alasan has been reported to be 4.6 g 1-1 while that of the SC14 has been estimated to be 3.9 g 1-1.

The reconstitution experiments revealed that the capsular exopolysaccharide fraction exhibited high emulsification activity alone pointing towards it's role in the overall emulsification. A minicapsule has been demonstrated in case of SC14. In an earlier report, Ac. calcoaceticus BD4, a highly encapsulated strain, was shown to produce an extracellular polysaccharide responsible for emulsifying activity. (Kaplan and Rosenberg 1982). The capsule of RAG-1 has been demonstrated to be a cell-bound form of the emulsifier, emulsan (Shabtai et al. 1985). Disappearance of the cellular capsule during stationary growth phase and it's subsequent release into the fermentation medium are attributable to the simultaneous maximum production of the bioemulsifier. All the fractions, viz, protein, polysaccharide, extracellular protein and exopolysaccharide, are essential for optimum bioemulsifier activity. Interestingly, 95.2% (1676.2 EU ml-1) reconstitution was achieved. Protein fraction of the BD4 emulsan was found to play a crucial role in emulsifying activity (Kaplan et al. 1987). On the contrary, in case of RAG-1 emulsan, protein was not absolutely required for emulsifying activity.

A phenomenon of bioemulsifier accumulation on cell surface during logarithmic growth phase and it's subsequent release into the medium during stationary growth phase is reported in case of emulsan (Rubinovitz et al. 1982; Shabtai et al. 1985; Shabtai and Gutnick 1985). Ac. junii SC14 also displays a similar trend. The pattern of esterase production by SC14 is unique. At 30 h of fermentation, at which maximum bioemulsifier production occurred, 61.3% of the total peak esterase activity was found associated with the cells and only 38.6% activity was detected in the cell-free broth. Role of esterase in release of emulsan from the cell surface of RAG-1 has been well established wherein a decline in cell-associated esterase was recorded to lead to a subsequent rise in the cell free esterase and in turn to increase in emulsan production. A fraction of cell free esterase was also demonstrated to be associated with the emulsan (Shabtai and Gutnick 1985). On the contrary, we report simultaneous increase in cell-associated and cell free esterase activity coincident with the maximum bioemulsifier production by SC14. Negligible activity was found associated with the bioemulsifier. Kinetics and distribution of esterase produced by SC14 suggest it's. involvement in the release of emulsifying factor from the cell surface into the fermentation medium. This is supported by our finding that cell pellet did not show detectable emulsifying activity (data not shown).

To summarize, the present work has demonstrated good emulsifying activity by Acinetobacter strains isolated from healthy human skin owing to the physico-chemical microenvironment of their habitat. On the basis of promising results obtained during the present study, healthy human skin appears to be a novel source of bacteria capable of bioemulsifier production. Findings of the present study would act as a foundation on which to be based similar investigations in the future.

TABLE 1
Effect of inducer oil on bioemulsifier production by
Acinetobacter junii SC14.

| Growth in | Emulsification of (U ml⁻¹) | | | |
|---|---|---|---|---|
| 1% of* | Almond oil | Castor oil | Olive oil | Palm oil |
| Almond oil | 116.6 | 72.0 | 66.4 | 85.0 |
| Castor oil | 66.1 | 97.1 | 69.1 | 79.0 |
| Olive oil | 99.5 | 85.6 | 87.1 | 85.1 |
| Palm oil | 90.3 | 79.1 | 80.1 | 86.1 |

*Fermentation medium supplemented with 1% test oil.

TABLE 2
Reconstitution of emulsification activity by isolated
fractions of the bioemulsifier produced by
Acinetobacter junii SC14.

| Fraction | % Activity | EU ml⁻¹ |
|---|---|---|
| Purified Emulsifier | 100 | 1759.8 |
| Bioemulsifier Protein (BEP)* | 78.4 | 1380.0 |
| Extracellular Protein (EP)† | 78.2 | 1377.2 |
| Bioemulsifier Polysaccharide (BEPS)‡ | 74.5 | 1312.2 |
| Exopolysaccharide (EPS)^{§} | 76.2 | 1342.5 |
| BEP + BEPS | 84.7 | 1473.9 |
| BEP + EP | 78.0 | 1372.7 |
| BEP + EPS | 81.0 | 1425.0 |
| BEPS + EPS | 74.4 | 1310.6 |
| BEP + BEPS + EP + EPS | 95.2 | 1676.2 |

*Protein fraction of the purified bioemulsifier prepared by hot phenol treatment.
†Extracellular protein obtained by 60% ammonium sulfate precipitation of cell-free culture broth.
‡Polysaccharide fraction from purified bioemulsifier isolated by water extraction of phenol phase
^{§}Capsular polysaccharide precipitate obtained by acetone precipitation of the homogenized culture supernatant.

We claim:

1. A bioemulsifier comprising of protein content 50.5%, polysaccharide 43% and lipid content 3.8%.

2. A bioemulsifier as claimed in claim 1 wherein the bioemulsifier showed peak esterase activity cell associated of order of 61.3% and 38.6% activity was secreted into the fermentation medium.

3. A bioemulsifier as claimed in claim. 1 wherein increase in bioemulsifier concentration from 0.5 ml to 3 ml against a fixed volume of 6 ml almond oil resulted in a reduction of viscosity of almond oil by 40.3%.

4. A bioemulsifier as claimed in claim 1 wherein the almond oil and water emulsion maintains its stability up to 90% up to 6 days at 37° C. cm 5. A bioemulsifier as claimed in claim 1 wherein the bioemulsifier retains 35% stability after 140 hours at 10° C.

6. A bioemulsifier as claimed in claim 1 is useful for preparing stable cosmetics (skin care product) and stable pharmaceutical ointment preparations.

7. A process for production of bioemulsifier from Acinetobacter strains which comprises:
(i) obtaining eight Acinetobacter strains from healthy human skin and two each from burn wounds and soil after enrichment of respective samples in enrichment medium,
(ii) identifying the isolates up to genus level as per the chromosomal DNA transformation assay,
(iii) growing soil bacterial isolates between 30° to 40° C. in a mineral salt medium supplemented with paptone and oil under shaking conditions at a rate around 150 rpm at a pH rangeing between 4 to 9 for a period of 2-3 days,
(iv) obtaining cell-free culture after centrifugation at about 8000 rev per minute for a period of about 20 minutes,
(v) adding chilled acetone to the broth and incubating at about 4° C. for about 15 hours,
(vi) collecting the precipitate by centrifugation, dissolving the precipitate in water and purifying by dialysis method, lyophilizing the dialysate to obtain the partially purified bioemulsifier.

8. A process as claimed in claim 7 wherein bacterial isolates used are 8 skin isolates selected and identified as Ac. baumannii, Ac. lwoffii, Ac. junii and remaining two as Ac. haemolyticus.

9. A process as claimed in claim 7 wherein the burn wounds isolates were Ac. baumannii.

10. A process as claimed in claim 7 wherein soil isolates were from Ac. calcoaceticus.

11. A process as claimed in claim 7 wherein the oil used is selected from olive oil, palm oil, almond oil, castor oil.

12. A process as claimed in claim 7 wherein the mineral medium used is comprising 0.5 g k₂HPO₄, 1 g NH₄Cl, 2 g Na₂SO₄, 2 g KNO₃, 0.2 g MgSO₄.7H₂O, and 0.002 g FeSO₄.7H₂O.

13. A process as claimed in claim 7 wherein Ac. junii SC14 isolated from healthy human skin exhibited maximum bioemulsifier production.

14. A process as claimed in claim 7 wherein the almond oil and water emulsion maintains its stability up to 90% at 37° C.

15. A process as claimed in claim 8 wherein the cell free supernatant of SC14 culture grown in presence of 18% almond oil exhibited 1759.8 EU ml⁻¹.

16. A process as claimed in claim 7 wherein yield of bioemulsifier is around 4 g L¹.

17. A process as claim in claim 7 wherein the bioemulsifier contains protein 50.5%, polysaccharide 43% and 3.8% lipid.

18. A process as claimed in claim 7 wherein 61.3% of peak esterase activity was observed to be cell associated and 38.6% activity was excreted into the fermentation medium.

19. A process as claimed in claim 7 wherein increase in bioemulsifier concentration from 0.5 ml to 3 ml against a fixed volume of almond oil resulted in a reduction of viscosity of almond oil by 40.3%.
